(19) United States
(12) Patent Application Publication
(10) Pub. No.: US 2010/0112606 A1
(43) Pub. Date: May 6, 2010

(54) MEASUREMENT AND ANALYSIS OF LEUKOTRIENES

(76) Inventors: Michael Armstrong, Aurora, CO (US); Nichole Ann-Marie Reisdorph, Centennial, CO (US); Nathan Rabinovitch, Denver, CO (US)

Correspondence Address: SHERIDAN ROSS PC, 1560 BROADWAY, SUITE 1200, DENVER, CO, 80202, US

(21) Appl. No.: 12/581,436

(22) Filed: Oct. 19, 2009

Related U.S. Application Data
(60) Provisional application No. 61/196,623, filed on Oct. 17, 2008.

(51) Int. Cl. G01N 33/542; C12Q 1/02; C12Q 1/26; H01J 49/26

(52) U.S. Cl. 435/7.92; 435/29; 435/25; 250/282

(57) ABSTRACT

The present invention provides a new analytical method for measuring leukotrienes in a clinical sample using liquid chromatography and tandem mass spectrometry (LCMSMS). The method provides a simple, rapid and low-cost assay for the measurement of leukotriene levels in a clinical sample with high accuracy and precision over the physiological range. The present invention further provides a method to determine the susceptibility of a subject to treatment with a leukotriene modifier, as wells as methods for diagnosis of a chronic obstructive disease of the airways and for predicting the risk of exacerbation of the same.

### CROSS-REFERENCE TO RELATED APPLICATIONS

This application claims the benefit of priority under 35 U.S.C. §119(e) from U.S. Provisional Application Ser. No. 61/196,623, filed Oct. 17, 2008, the contents of which are incorporated herein in their entirety by this reference.

### GOVERNMENT SUPPORT

This invention was supported in part with funding provided by NIH grants K23 ES015510-01 and U01-HL-81335, awarded by the National Institute of Health. The government has certain rights to this invention.

### FIELD OF THE INVENTION

The field of the present invention is measurement and analysis of Leukotriene. such as Leukotriene E4 (LTE₄), in a biosample and its relevance to diagnosis and therapies for chronic obstructive diseases of the airways.

### BACKGROUND OF THE INVENTION

Lipid mediators have emerged as key mediators of asthmatic inflammation and specific targets for asthma therapy to improve symptoms, lung function, and disease control. The best characterized in the context of asthma, the cysteinyl leukotrienes (i.e., LTC₄, LTD₄, LTE₄: “cysLT”), are potent mediators of bronchoconstriction and inflammation in asthma. Although inhibitors of pro-inflammatory lipid mediators are common therapeutic options for asthma, their clinical role is not clearly established because of the variability in response to therapy (Malmstrom 1999; Zhang 2002; Szefler 2005) and a lack of good, commercialized predictors to distinguish therapeutic responders from non-responders.

Elevated urine LTE₄ levels (the stable, urine-excreted metabolite of the cysLTs) are associated with an increased likelihood of clinical improvement in asthma with leukotriene receptor antagonist (LTRA) treatment (Szefler 2005; Rabinovitch 2006; Cai 2007). In one study, a threshold urine LTE₄ level was associated with clinical improvement with LTRA treatment (Szefler 2005); in another study, persistent LTE₄ elevation was a better correlate (Rabinovitch 2006). In a more recent study, it was proposed that a ratio of LTE₄ to fractional exhaled nitric oxide (FENO) could be used to predict responsiveness (Rabinovitch 2008). These studies indicate a potential role for urine LTE₄ measures to guide institution of daily LTRA as both first-line and add-on controller therapy in children with asthma.

In all three studies, however, a strong prediction threshold could not be identified. This shortfall was likely due to problems with the reliability and reproducibility of the assay used for measuring the LTE₄ levels in the urine. Improving the LTE₄ assay would lead to a strong prediction threshold to guide institution of leukotriene modifier therapy in asthma.

Further, the measurement of LTE₄ in urine is a noninvasive method to assess changes in the rate of cysteinyl leukotriene (CysLT) production and excretion (Kumlin 2000, Smith 1992, Westcott 1998). Although CysLTs are known to be key mediators and modulators in the pathogenesis of chronic obstructive diseases of the airways, such as asthma (Doucet 1990, Gyllfors 2005, Busse 2005), there have been relatively few clinical studies assessing the role of LTE₄ as an asthma-related biomarker and none that have determined relevant cut-off points which would allow a clinician to utilize this test to diagnose or manage asthma. The paucity of such studies are partially related to technical limitations in measuring LTE₄ making this a difficult assay to use in the clinical laboratory setting.

In general, currently two basic methods of measuring LTE₄ are employed. These are antibody-based, and mass spectrometry-based.

Measurement of LTE₄ using immunoassay: The LTE₄ immunoassay is based on the competition between free LTE₄ in the sample and an LTE₄-acetylcholinesterase (AChE) conjugate for a limited amount of LTE₄ antiserum. Because the concentration of the LTE₄ tracer is held constant while the concentration of the LTE₄ varies, the amount of LTE₄ tracer that is able to bind to the LTE₄ antiserum will be inversely proportional to the concentration of the LTE in the sample. This antibody-LTE₄ complex binds to a mouse monoclonal anti-rabbit IgG that has been previously attached to well of a microtiter plate. A substrate for the AChE is added and the product of this enzymatic reaction has a yellow color, the intensity of which can be measured spectrophotometrically. The intensity of the color is proportional to the amount of LTE₄ tracer bound to the well and is inversely proportional to the amount of free LTE₄ in the sample.

However, urine contains impurities which can interfere with the assay. To eliminate contaminants from the urine, it is often subjected to an affinity purification step prior to its use in the immunoassay. The accuracy of these methods is dependent on the specificity of the chosen antibody. Further, there is wide variation among extraction methods and efficiencies. One such method utilizes crude urine (Kumlin 1995) and another HPLC for separation of various leukotrienes (Westcott 1990), both followed by enzyme immunoassay (EIA). Another strategy employs a peptidoleukotriene immunoaffinity resin to reportedly simplify extraction (Westcott 1998). An EIA kit is distributed by Cayman Chemical Company has been utilized in clinical studies of children with asthma (Mai 2005).

Measurement of LTE₄ using mass spectrometry: A number of mass spectrometry based methods to measure LTE₄ levels in urine have been previously employed, many of which employ multiple reaction monitoring (MRM) with a triple quadrupole (QQQ) mass spectrometer. MRM is used to specifically filter for a molecule of interest in the first quadrupole, fragment that molecule in the second quadrupole, then filter for only a single resulting fragment peak for that molecule. Fragmentation, or transitions, are also reproducible. LTE₄, for example, undergoes a transition from 440.2→301.2 m/z when fragmented due to the loss of the cysteinyl group and a water. Because a labeled analog of the molecule of interest will undergo identical fragmentation (i.e. MRM transitions), the peak area of the labeled molecule can be directly compared to the peak area of the endogenous, unlabeled molecule, providing quantitative information.

When a calibration curve and labeled internal standards are used, this method is often thought to be more precise than antibody-based assays, which are sometimes complicated by non-specific interactions between the antibody and other molecules.

However, these methods rely on complex up-front enrichment strategies which makes them less suitable for adaptation to a clinical assay. For example, Wu et al (1996) extracted LTE₄ and LTE₄-d3 with Empore membrane disks followed by analysis using a MRM strategy. Hardy, et al (2005) used solid phase extraction (SPE) followed by MRM. Kishi et al (2001) sought to improve the efficiency and speed of these methods through the use of on-line extraction of LTE₄ followed by MRM.

Although the existing techniques have some merit, none of them have been accepted as a standardized approach and it is not clear which method provides the highest degree of sensitivity, accuracy and precision. Further, critics of the LTE₄ immunoassays cite concerns over sample loss during the sample preparation phase as well as cross-reactivity of the antibody resulting in overestimation of LTE₄ levels (Kishi 2001). Conversely, mass spectrometry-based assays may also result in significant sample loss during sample preparation and due to the instability of the molecule (Kishi 2001).

Thus, none of the current methods are suitable for clinical purposes and there exists a need in the art for a specific, accurate, precise and simple assay for the measurement and analysis of LTE₄ in clinical samples.

### SUMMARY OF THE INVENTION

In one embodiment, the present invention includes a method for determining the level of a leukotriene present in a sample from a donor, comprising the steps of subjecting the sample to high performance liquid chromatography (HPLC) to partially purify the sample, and subjecting the partially purified sample to tandem mass spectrometry. In various embodiments, the leukotriene is selected from the group consisting of LTA₄, LTB₄, LTC₄, LTD₄, LTE₄, and LTF₄.

In another embodiment, the present invention includes a method of determining the susceptibility of a donor to treatment with a leukotriene modifier. This method comprises obtaining a sample from the donor and determining the level of leukotriene in the sample selected from the group consisting of LTA₄, LTB₄, LTC₄, LTD₄, LTE₄, and LTF₄, and combinations thereof, wherein presence of the leukotriene in the sample at an elevated level as compared to a baseline level established from a control sample, identifies the donor as susceptible to treatment with the leukotriene modifier. In some embodiments, the step of determining the level of leukotriene in the sample comprises determining the level of LTE₄. In some embodiments, the leukotriene modifier is selected from the group consisting of a leukotriene receptor antagonist and a leukotriene synthesis inhibitor. In some embodiments, the leukotriene receptor antagonist is selected from the group consisting of montelukast, zafirlukast and pranlukast. In some embodiments, the leukotriene modifier is an inhibitor of the 5-lipoxygenase pathway of leukotriene metabolism. In some embodiments, the leukotriene modifier inhibits the activity of 5-lipoxygenase. In some embodiments, the leukotriene modifier inhibits the activity of 5-lipoxygenase-activating protein (FLAP). In some embodiments, the leukotriene modifier is Zileuton. In some embodiments, the donor has, or is at risk of developing a chronic obstructive disease of the airways. Examples of such a disease include, without limitation, asthma, occupational asthma, exercise-induced asthma, pollution-induced asthma, allergic bronchopulmonary aspergillosis, eosinophilic pneumonia, allergic bronchitis bronchiectasis, hypersensitivity pneumonitis, reactive airway disease syndrome, interstitial lung disease, hyper-eosinophilic syndrome, rhinitis, sinusitis, and parasitic lung disease. In some embodiments, the chronic obstructive disease of the airways is asthma and the level of LTE₄ in the sample is at or greater than about 80 pg/mg of creatinine.

In another embodiment, the present invention includes a method for diagnosing a chronic obstructive disease of the airways in a donor. This method comprises obtaining a sample from the donor and determining the level of leukotriene in the sample selected from the group consisting of LTA₄, LTB₄, LTC₄, LTD₄, LTE₄, and LTF₄, and combinations thereof, wherein presence of leukotriene in the sample at an, elevated level as compared to a baseline level established from a control sample, identifies the donor as having or likely to develop the chronic obstructive disease of the airways. In some embodiments, the step of determining the level of leukotriene in the sample comprises determining the level of LTE₄. In some embodiments, the chronic obstructive disease of the airways is asthma and the level of LTE₄ in the sample is at or greater than about 30 pg/mg of creatinine.

In another embodiment, the present invention includes a method for predicting the risk for a donor of exacerbation due to a chronic obstructive disease of the airways. This method comprises obtaining a sample from the donor and determining the level of leukotriene in the sample selected from the group consisting of LTA₄, LTB₄, LTC₄, LTD₄, LTE₄, and LTF₄, and combinations thereof, wherein presence of leukotriene in the sample at an elevated level as compared to a baseline level established from a control sample, identifies the donor as at risk of exacerbation due to the chronic obstructive disease. In some embodiments, the step of determining the level of leukotriene in the sample comprises determining the level of LTE₄. In some embodiments, the chronic obstructive disease of the airways is asthma and the level of LTE₄ in the sample is at or greater than about 90 pg/mg of creatinine.

In various embodiments, the step of determining the level of LTE₄ in the sample comprises the method of the present invention comprising the steps of subjecting the sample to high performance liquid chromatography (HPLC) to partially purify the sample and subjecting the partially purified sample to tandem mass spectrometry.

In some embodiments, the HPLC system comprises an enrichment column and an analytical column, wherein the two columns are connected to each other by a switching valve, the analytical column is connected to a mass spectrometer, and the effluent from the analytical column is directed into the mass spectrometer as the leukotriene elutes from the analytical column.

In some embodiments, the mass spectrometer comprises more than one quadrupole; leukotriene molecules with a specific mass to charge ratio (“m/z ratio”) are selected in the first quadrupole of the mass spectrometer and are dissociated to form leukotriene fragments in the second quadrupole; and one leukotriene fragment is selected for quantitative analysis while another leukotriene fragment is used for qualitative analysis.

In some embodiments, the leukotriene is LTE₄ and the leukotriene molecules selected in the first quadrupole have a m/z ratio in positive mode of about 440.2 and a m/z ratio in negative mode of about 438.2. In further embodiments, the LTE₄ fragments comprise fragments of m/z ratios of about 189.2, 205.2, 265.2, 283.2, and 301.2 Da in positive mode and about 235.2, 289.2, 317.2, 333.2, 351.2 and 420 Da in negative mode. In some embodiments, in positive mode the LTE₄ fragment of m/z ratio of 301.2 is selected for quantitative analysis and the LTE₄ fragment of m/z ratio of 189.2 is selected for qualitative analysis. In some embodiments, in negative mode the LTE₄ fragment of m/z ratio of 333.2 is selected for quantitative analysis and the LTE₄ fragment of m/z ratio of 351.2 is selected for qualitative analysis.

In some embodiments, the pH of the HPLC solvent is in the range of about 2.5 to about 8.5. In some embodiments, a known amount of labeled leukotriene is added to the sample. In some embodiments, the labeled leukotriene is labeled with a stable isotope. In some embodiments, prior to HPLC, the sample is centrifuged and the supernatant is subjected to HPLC. In various embodiments, the sample may be urine, blood, saliva, sputum, broncho-alveolar fluid or exhaled breath condensate.

### BRIEF DESCRIPTION OF THE DRAWINGS

FIG. 1 is schematic representation of the HPLC system illustrating the valve and pump orientation for the on-line purification of LTE₄ in urine.

FIG. 2 is a schematic representation of the MRM during tandem mass spectrometry.

FIG. 3a and FIG. 3b show the calibration curve for LTE₄ in water from 5 pg/ml to 500 pg/ml.

FIGS. 4A and 4B illustrate that LTE₄ levels are significantly higher in children with asthma. The two boxes in FIG. 4A represent log-transformed LTE₄ levels controlled for dilution (pg/mg creatinine) for the asthmatic and control groups. Midline box represents the mean level with box edges at 25^{th} and 75^{th} percentile and whiskers at 95^{th} percentile. P-values calculated from unpaired t-test. FIG. 4B shows the Receiver Operator Characteristic (ROC) for the relationship between LTE₄ and asthma diagnosis.

### DETAILED DESCRIPTION OF THE INVENTION

The present invention is directed to a method to determine the level of leukotrienes present in a sample. The method of the invention provides excellent sensitivity, precision, and accuracy for the rapid measurement of leukotrienes present in a sample, does not require labor intensive and costly purification procedures, and is suitable for adaptation to clinical samples. The present invention is further directed to clinical methods for determining the susceptibility of a donor to treatment with a leukotriene modifier, for diagnosing a chronic obstructive disease of the airways, and for predicting the risk of such disease.

In one embodiment, the present invention includes a method for determining the level of a leukotriene present in a sample from a donor. The leukotriene may be LTA₄, LTB₄, LTC₄, LTD₄, LTE₄, or LTF₄, or any combination thereof. The method comprises subjecting the sample to high performance liquid chromatography (HPLC) to partially purify the sample and subjecting the partially purified sample to tandem mass spectrometry.

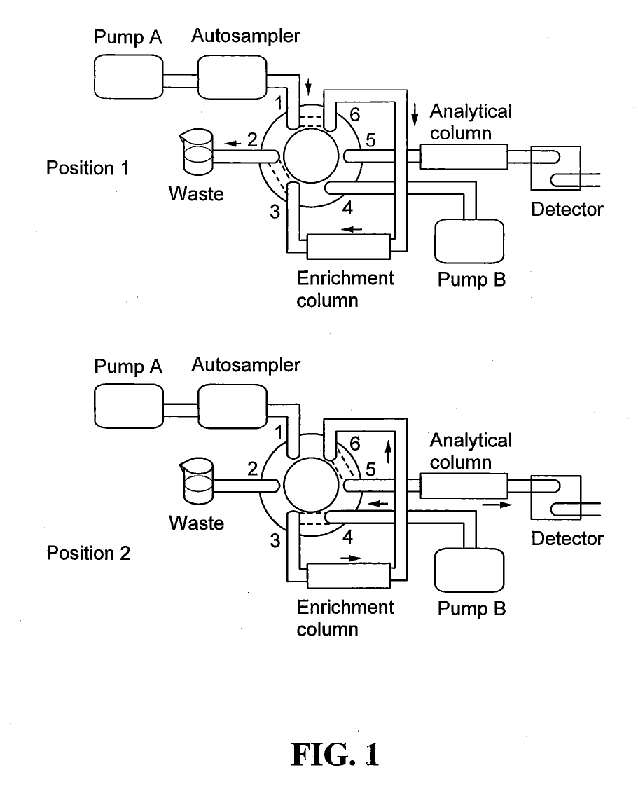

The method utilizes a system that comprises an HPLC system connected to tandem mass spectrometer. This system is also referred to in this application as the “LCMSMS system” and the method is referred to as the “LCMSMS method.” In one embodiment, the HPLC system comprises an enrichment column and an analytical column. The two columns are connected to each other by means of a switching valve. The switching valve enables the system to change the flow of liquid from one column to the other. The system further comprises two pumps for applying solvents to the columns. The pumps are capable of forming separate gradients. The analytical column is further connected to a mass spectrometer. A particular embodiment of this system is shown in FIG. 1.

As shown in FIG. 1, the system may be equipped with an autosampler for loading the sample onto a column. In a preferred embodiment, the sample may be injected from the autosampler, through the switching valve, onto the enrichment column for concentration/purification purposes. This is illustrated in FIG. 1, position 1. Upon loading, the leukotriene molecules in the sample become bound to the enrichment column, and the solvent along with the unbound non-leukotriene solutes flows from the enrichment column, through the switching valve, to waste. During this time, Pump B pumps a solvent through the switching valve, to the analytical column; the solvent through the analytical column may be directed to waste by means of a divert valve (not shown in FIG. 1).

In some embodiments, the HPLC solvent may comprise an aqueous solvent and one or more organic solvents selected from the group consisting of acetonitrile, methanol, and isopropanol. In the enrichment column, the one or more organic solvents may be present at a concentration gradient of about 5% to about 40% and in the analytical column, the one or more organic solvents may be present at a concentration gradient of about 10% to about 50%. In various embodiments, the pH of the HPLC solvent may range from about 2 to about 9, from about 2.5 to about 8.5 and from about 3 to about 8.

The switching valve can be turned in such a way that Pump B begins to pump solvent through the enrichment column, which is directed through the switching valve to the analytical column. This is illustrated in FIG. 1, position 2. At this time, Pump A is not connected to either column and pumps directly through the switching valve to waste. As pump B gradually pumps more solvent, at a certain concentration of the solvent, the leukotriene molecules move from the enrichment column to the now-connected analytical column. As the leukotriene molecules pass through the analytical column, further resolution of leukotriene can be achieved.

The analytical column is further connected to a tandem mass spectrometer. The column effluent from the analytical column comprising the leukotriene molecules flows into the mass spectrometer as the leukotriene molecules are eluting from the analytical column. Upon entry into the mass spectrometer, the leukotriene molecules are ionized or charged. The ionizing agent may comprise ammonium hydroxide, ammonium acetate, acetic acid, formic acid, trifluoracetic acid or a combination thereof.

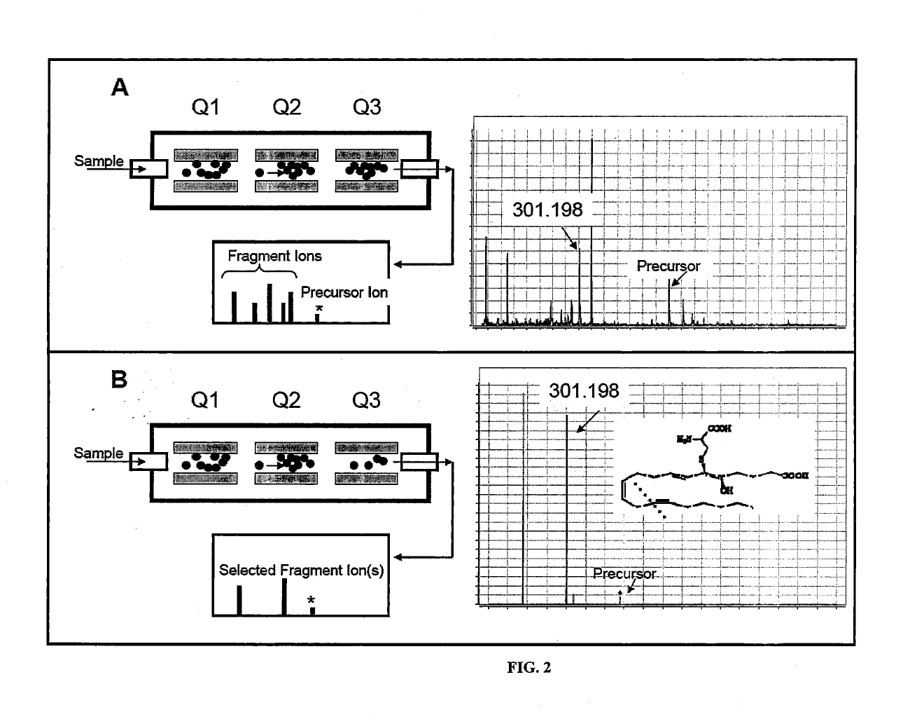

In one embodiment, illustrated in FIG. 2, the tandem mass spectrometer comprises three quadrupoles. In the first quadrupole, Multiple Reaction Monitoring (“MRM”) is used to specifically filter for a leukotriene molecule with a specific mass to charge ratio (“m/z ratio”). In the second quadrupole, the leukotriene molecule is fragmented using collision induced dissociation which is induced by gas molecules such as helium, nitrogen, or argon. The third quadrupole is used to filter for a single resulting fragment of interest. One leukotriene fragment may be selected for quantitative analysis and another leukotriene fragment may be used for qualitative analysis.

In one embodiment, a known amount of labeled leukotriene is added to the sample prior to loading the sample onto the HPLC. Because a labeled analog of the molecule of interest will undergo identical fragmentation (i.e. MRM transitions), the peak area of the labeled molecule can be directly compared to the peak area of the unlabeled molecule in the sample, providing quantitative information. The label may be a stable isotope. In some embodiments, the sample is centrifuged and the supernatant is subjected to HPLC.

An embodiment of the method of the present invention is described in Example 1. Although this example describes determining the level of LTE₄, one skilled in the art will understand that the method of the present invention is applicable to measuring other leukotrienes.

LTE₄ with m/z ratio of 440.2 in positive mode and 438.2 in negative mode is selected in the first quadrupole of the mass spectrometer. Upon fragmentation in the second quadrupole the LTE₄ fragments comprise fragments of m/z ratios of approximately 189.2, 205.2, 265.2, 283.2, and 301.2 Da in positive mode and approximately 235.2, 289.2, 317.2, 333.2, 351.2 and 420 Da in negative mode. In one embodiment, in positive mode the LTE₄ fragment of m/z ratio of 301.2 is selected for quantitative analysis and the LTE₄ fragment of m/z ratio of 189.2 is selected for qualitative analysis. In another embodiment, in negative mode the LTE₄ fragment of m/z ratio of 333.2 is selected for quantitative analysis and the LTE₄ fragment of m/z ratio of 351.2 is selected for qualitative analysis.

As described in detail in Example 1, the present method has excellent sensitivity, precision, and accuracy for the measurement of LTE₄ in a sample. The method is linear along a physiological range, is fairly rapid (15 minutes), requires no sample preparation beyond a simple centrifugation, and is suitable for adaptation to clinical samples. Further, a comparison of ELISA and the LCMSMS method of the present invention demonstrated that the present method is superior to the ELISA method (Example 2). The ELISA method leads to higher estimations of LTE₄, as well as high coefficients of variance (“CV”). The lower variance reported using the present methods allows clinical investigators to better differentiate between patient phenotypes.

In addition to minor sample loss following freeze/thaw cycles, samples were relatively stable when stored at 4° C. for up to 7 days. Results from sample integrity studies (Example 3) may be used to determine shipping and storage conditions.

In another embodiment, the present invention includes a method of determining the susceptibility of a donor to treatment with a leukotriene modifier. The method comprises obtaining a sample from the donor and determining the level of leukotriene in the sample. The leukotriene may be LTA₄, LTB₄, LTC₄, LTD₄, LTE₄, or LTF₄, or any combination thereof. The presence of the leukotriene in the sample at an elevated level as compared to a baseline level established from a control sample, identifies the donor as being susceptible to treatment with the leukotriene modifier. In some embodiments, the donor may have or may be at a risk of developing a chronic obstructive disease of the airways and the treatment with a leukotriene modifier may be provided to treat the disease.

A leukotriene modifier includes any agent that modifies or inhibits the inflammatory activities of leukotrienes. Such modifications or inhibitions can take place at a variety of levels. For example, the inflammatory activity of a leukotriene can be modified or inhibited by modifying or inhibiting leukotriene metabolism. Leukotriene metabolism can be effectuated by inhibition of 5-lipoxygenase or inhibition of 5-lipoxygenase-activating protein (FLAP) (e.g., Zileuton also known as ZYFLO® Abbott Laboratories, Abbott Park, Ill.). The inflammatory activity of a leukotriene can alternatively be modified or inhibited by modifying or inhibiting biological functioning of leukotrienes. For example, inhibition of the biological functioning of leukotrienes can be effectuated by agents that antagonize the actions of leukotriene receptors (also known as leukotriene receptor antagonists or LTRAs). LTRAs include but are not limited to montelukast, zafirlukast (e.g. ACCOLATE® AstraZeneca, Wilmington Del.) and pranlukast. The biological functioning of leukotrienes can also be inhibited by interference with receptor binding such as by an antibody to a leukotriene.

In another embodiment, the present invention includes a method for diagnosing a chronic obstructive disease of the airways in a donor. The method comprises obtaining a sample from the donor and determining the level of leukotriene in the sample. The leukotriene may be LTA₄, LTB₄, LTC₄, LTD₄, LTE₄, or LTF₄, or any combination thereof. The presence of the leukotriene in the sample at an elevated level as compared to a baseline level established from a control sample, identifies the donor as having or likely to develop the chronic obstructive disease of the airways.

In a further embodiment, the present invention includes a method for predicting the risk of exacerbation of a chronic obstructive disease of the airways for a donor. The method comprises obtaining a sample from the donor and determining the level of leukotriene in the sample. The leukotriene may be LTA₄, LTB₄, LTC₄, LTD₄, LTE₄, or LTF₄, or any combination thereof. The presence of the leukotriene in the sample at an elevated level as compared to a baseline level established from a control sample, identifies the donor as being at risk of exacerbation due to the chronic obstructive disease.

In some embodiments, the step of determining the level of leukotriene in the sample comprises determining the level of LTE₄. The level of LTE₄ may be determined using the LCMSMS method of the present invention. One way of expressing the level of LTE₄ is in terms of picograms of LTE₄ per milligram of creatinine present in the sample.

Examples of a chronic obstructive disease include, without limitation, asthma, occupational asthma, exercise-induced asthma, pollution-induced asthma, allergic bronchopulmonary aspergillosis, eosinophilic pneumonia, allergic bronchitis bronchiectasis, hypersensitivity pneumonitis, reactive airway disease syndrome, interstitial lung disease, hyper-eosinophilic syndrome, rhinitis, sinusitis, and parasitic lung disease. In a preferred embodiment, the chronic obstructive disease is asthma.

According to the present invention, a “baseline level” is a normal level of LTE₄ against which the level of LTE₄ in the sample is compared. Based on the control or baseline level of LTE₄, it is determined whether a sample has an increased or elevated, decreased, or substantially the same LTE₄ level. The term “negative control” or “normal control” used in reference to a baseline level of LTE₄ typically refers to a baseline level established in a sample from the subject or from a population of individuals which is believed to be normal (i.e., non-disease). A baseline can also be indicative of a positive diagnosis of the disease; such a baseline level is referred to as a “positive control” baseline and refers to a level of LTE₄ established in a sample from the donor, another donor or a population of donors, wherein the donor was believed to be diseased.

The baseline level of LTE₄ may be established from control samples, and preferably control samples that were obtained from a population of matched individuals. The phrase “matched individuals” refers to a matching of the control individuals on the basis of one or more characteristics which are suitable for the disease to be evaluated. For example, control individuals can be matched with the subject to be evaluated on the basis of gender, age, race, or any relevant biological or sociological factor that may affect the baseline of the control individuals and the subject (e.g., preexisting conditions, consumption of particular substances, levels of other biological or physiological factors). To establish a control or baseline level of LTE₄, samples from a number of matched individuals are obtained and evaluated for LTE₄ levels. The sample type is preferably of the same sample type as the sample type to be evaluated in the donor. The number of matched individuals from whom control samples must be obtained to establish a suitable control level (e.g., a population) can be determined by those of skill in the art, but should be statistically appropriate to establish a suitable baseline for comparison with the subject to be evaluated (i.e., the test subject). The values obtained from the control samples are statistically processed using any suitable method of statistical analysis to establish a suitable baseline level using methods standard in the art for establishing such values.

It will be appreciated by those of skill in the art that a baseline need not be established for each assay as the assay is performed but rather, a baseline can be established by referring to a form of stored information regarding a previously determined baseline level of LTE₄ for a given control sample. Such a form of stored information can include, for example, but is not limited to, a reference chart, listing or electronic file of population or individual data regarding “normal” (negative control) or disease positive LTE₄ level; a medical chart for the subject recording data from previous evaluations; or any other source of data regarding baseline LTE₄ level that is useful.

As described in detail in Examples 4 and 5, the present inventors utilized the LCMSMS method of the present invention for measuring LTE₄ level in a sample and determined clinically relevant cut-off values for LTE₄ that can be used to diagnose asthma and/or predict exacerbation due to asthma.

Accordingly, in one embodiment, the method of the present invention includes a method for diagnosing a chronic obstructive disease in a donor. The method comprises obtaining a sample from the donor and determining the level of LTE₄ in it. Presence of LTE₄ in the sample at a level greater than 15 pg/mg, 20 pg/mg, 21 pg/mg, 22 pg/mg, 23 pg/mg, 24 pg/mg, 25 pg/mg, 26 pg/mg, 27 pg/mg, 28 pg/mg, 29 pg/mg, 30 pg/mg, 31 pg/mg, 32 pg/mg, 33 pg/mg, 34 pg/mg, 35 pg/mg, 36 pg/mg, 37 pg/mg, 38 pg/mg, 39 pg/mg, 40 pg/mg, 41 pg/mg, 42 pg/mg, 43 pg/mg, 44 pg/mg, 45 pg/mg, 50 pg/mg, 55 pg/mg, 60 pg/mg, 65 pg/mg, 70 pg/mg, 75 pg/mg, 80 pg/mg, 85 pg/mg, 90 pg/mg, 95 pg/mg, 100 pg/mg, 150 pg/mg, 200 pg/mg, 300 pg/mg, 400 pg/mg, 500 pg/mg, 1000 pg/mg, 1500 pg/mg, or 2000 pg/mg, (standardized per milligram of creatinine) identifies the donor as having or likely to develop the chronic obstructive disease.

In another embodiment, the method of the present invention includes a method for predicting the risk of exacerbation for a donor due to a chronic obstructive disease of the airways. This method, comprises obtaining a sample from the donor and determining the level of LTE₄ in it. Presence of LTE₄ in the sample at a level greater than 70 pg/mg, 75 pg/mg, 76 pg/mg, 77 pg/mg, 78 pg/mg, 79 pg/mg, 80 pg/mg, 81 pg/mg, 82 pg/mg, 83 pg/mg, 84 pg/mg, 85 pg/mg, 86 pg/mg, 87 pg/mg, 88 pg/mg, 89 pg/mg, 90 pg/mg, 91 pg/mg, 92 pg/mg, 93 pg/mg, 94 pg/mg, 95 pg/mg, 96 pg/mg, 97 pg/mg, 98 pg/mg, 99 pg/mg, 100 pg/mg, 101 pg/mg, 102 pg/mg, 103 pg/mg, 104 pg/mg, 105 pg/mg, 110 pg/mg, 115 pg/mg, 120 pg/mg, 125 pg/mg, 130 pg/mg, 140 pg/mg, 150 pg/mg, 160 pg/mg, 170 pg/mg, 180 pg/mg, 190 pg/mg, 200 pg/mg, 250 pg/mg, 300 pg/mg, 350 pg/mg, 400 pg/mg, 450 pg/mg, 500 pg/mg, 1000 pg/mg, 1500 pg/mg, or 2000 pg/mg, identifies the donor as at risk of exacerbation due to the chronic obstructive disease. Exacerbation refers to an increase in the severity of a disease or its symptoms, and may require hospitalization of the donor due to the disease.

In another embodiment, the method of the present invention includes a method of determining the susceptibility of a donor to treatment of asthma with a leukotriene modifier. This method comprises obtaining a sample from the donor and determining the level of LTE₄ in it. Presence of LTE₄ in the sample at a level greater than 60 pg/mg, 65 pg/mg, 66 pg/mg, 67 pg/mg, 68 pg/mg, 69 pg/mg, 70 pg/mg, 71 pg/mg, 72 pg/mg, 73 pg/mg, 74 pg/mg, 75 pg/mg, 76 pg/mg, 77 pg/mg, 78 pg/mg, 79 pg/mg, 80 pg/mg, 81 pg/mg, 82 pg/mg, 83 pg/mg, 84 pg/mg, 85 pg/mg, 86 pg/mg, 87 pg/mg, 88 pg/mg, 89 pg/mg, 90 pg/mg, 91 pg/mg, 92 pg/mg, 93 pg/mg, 94 pg/mg, 95 pg/mg, 100 pg/mg, 105 pg/mg, 110 pg/mg, 115 pg/mg, 120 pg/mg, 125 pg/mg, 130 pg/mg, 140 pg/mg, 150 pg/mg, 160 pg/mg, 170 pg/mg, 180 pg/mg, 190 pg/mg, 200 pg/mg, 250 pg/mg, 300 pg/mg, 350 pg/mg, 400 pg/mg, 450 pg/mg, 500 pg/mg, 1000 pg/mg, 1500 pg/mg, or 2000 pg/mg, identifies the donor being susceptible to the treatment with a leukotriene modifier.

As used herein the term donor refers to any animal subject, and particularly, any vertebrate mammal, including, but not limited to, primates, rodents, livestock and domestic pets. Preferred mammals for the methods of the present invention include humans.

As used herein the term sample refers to any biological fluid sample obtained from the donor. This includes without limitation, urine, blood, saliva, sputum, broncho-alveolar fluid or exhaled breath condensate. In a preferred embodiment, the sample is urine.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims. The following examples are provided for the purpose of illustration and are not intended to limit the scope of the present invention. Each publication, sequence or other reference disclosed below and elsewhere herein is incorporated herein by reference in its entirety, to the extent that there is no inconsistency with the present disclosure.

### EXAMPLES

### Example 1

Example 1 illustrates the method for measurement of LTE₄ in a urine sample.

LTE₄ and LTE₄-d3 standards were purchased from Cayman Chemical (Ann Arbor, Mich.) or Biomol (Plymouth meeting, PA). LTE₄ ELISA and Affinity sorbent were also purchased from Cayman Chemical (Ann Arbor, Mich.). Water (HPLC grade) and Acetonitrile (UV) used for HPLC mobile phases was obtained from Burdick and Jackson (Morristown, N.J.). Acetic acid and ammonium hydroxide were obtained from Fisher Scientific (Fair Lawn, N.J.).

Human urine from healthy and asthmatic volunteers was collected in 120 ml urine collection vesicles. Immediately after sample collection, urine was placed in a 15-45 ml falcon tube and centrifuged at 3,000×g, for 10 minutes at 4° C. to remove any particulates. The supernatant was collected and placed in a new falcon tube or subaliquoted into microfuge tubes and frozen at −80° C. (Alternatively, samples were kept at 4° C., room temperature, or 30 degrees for stability assessment experiments described in example 2.) Human subject participation and sample collection were in accordance with IRB requirements.

An internal standard spike solution was prepared at a concentration of 1 ng/ml LTE₄-d3 in 50:50 Methanol:Water in 0.1% acetic acid and 0.036% ammonium hydroxide. Calibration solutions of LTE₄ were prepared from stock solutions at concentrations ranging from 5 to 1000 pg/ml. Reported physiological concentrations of LTE₄ are 10 to 60 pg/ml in normal subjects and up to several hundred pg/ml in some asthmatic patients.

800 μL of urine sample was placed in an autosampler vial along with 200 μL of internal standard spike solution giving a final concentration of 200 pg/ml of LTE₄-d3. The sample was vortexed for 5 seconds and loaded directly on to the LCMSMS system using high performance liquid chromatography (HPLC) as outlined below. FIG. 1 provides a schematic representation of the HPLC.

Liquid chromatography was carried out using an Agilent 1200 series HPLC equipped with a quaternary pump (pump “A”), a binary pump (pump “B”), and an autosampler with thermostat (Agilent Technologies, Palo Alto, Calif.). The HPLC system was equipped with an autosampler for loading the sample onto a column and the two pumps for pumping solvent through the column. Each pump was capable of forming a separate gradient. In the particular embodiment described here, both pumped the same buffer system; buffer A was 0.02% acetic acid in HPLC water adjusted to pH 5.6 with ammonium hydroxide (about 0.007% ammonium hydroxide), and buffer B was 0.02% acetic acid and 0.007% ammonium hydroxide in 100% acetonitrile. The HPLC system consisted of two columns, an enrichment column and an analytical column, and the two columns were connected through a switching valve. This switching valve enabled the system to change the flow of liquid from one column to the other. The pumps moved the solvent separately through the columns, but were both connected to the switching valve. The enrichment column was an Extend C18 4.6×12.5 mm 5 uM guard cartridge (Agilent Technologies, Palo Alto, Calif.). The Analytical column was an Agilent Eclipse C8 column (2.1×50 mm) with a 1.8 uM particle size fitted with an Eclipse C8 2.1×12.5 mm 5 uM guard column operated at 40° C.

900 μL of the prepared urine (including labeled LTE₄-d3) was injected from the autosampler onto the enrichment column, through pump A, for concentration/purification purposes, followed by valve switching and subsequent separation on the analytical column using the gradient conditions in table 1. Flow rates were 1 ml/min for pump A and 0.15 ml/min for pump B.

TABLE 1

| | | |
|---|---|---|
| Time | Buffer A | Buffer B |

Pump A

| | | |
|---|---|---|
| 0 | 95 | 5 |
| 1 | 95 | 5 |
| 1.01 | 70 | 30 |
| 4.5 | 70 | 30 |
| 4.51 | 0 | 100 |
| 7 | 0 | 100 |
| 7.01 | 95 | 5 |
| 14 | 95 | 5 |

Pump B

| | | |
|---|---|---|
| 0 | 70 | 30 |
| 3 | 70 | 30 |
| 3.01 | 65 | 35 |
| 4.5 | 65 | 35 |
| 11 | 62 | 38 |
| 14 | 62 | 38 |

Pump A started at 5% Buffer B (and 95% Buffer A) and remained at 5% Buffer B for one minute while the sample loaded onto the column and the LTE₄ molecules became bound to the column particles. The solvents and non-retained solutes flowed from the column, through the switching valve, to waste. At this time, Pump B pumped solvents through the switching valve through the analytical column which was connected to a mass spectrometer. However, at this point the flow of the solvent from Pump B through the column was directed to waste using a divert valve (not shown in FIG. 1) before the mass spectrometer. Following sample loading, Pump A switched immediately to 30% Buffer B at 1.01 minute and remained at 30% Buffer B, during which time, non-LTE₄ molecules were flushed to waste, while LTE₄ remained bound to the column. At 3 minutes, the switching valve turned and Pump B began to pump liquid through the enrichment column, then through the switching valve, and then through the analytical column. At this time, Pump A pumped directly through the switching valve to waste and was not connected to either column. Pump B gradually pumped more Buffer B through the enrichment and analytical columns. At a precise amount/percent of Buffer B, LTE₄ molecules moved from the enrichment column to the now-connected analytical column. The LTE₄ molecules were further resolved on the analytical column.

Detection of LTE₄ was accomplished using an Agilent 6410 triple quadrupole (QQQ) mass spectrometer (MS) coupled to a positive electrospray ionization source. Heated (300° C.) drying gas flowing at 10 L/min, with a nebulizer pressure of 15 PSIG, was used for droplet desolvation. Spray was induced with a capillary voltage of 4000V. The optimal fragmentor voltage of 80V and collision energy of 8V was determined by flow injection analysis. The QQQ was tuned and calibrated using Agilent G1969-85000 calibration and tuning mix (Agilent Technologies, Palo Alto, Calif.). During MRM, the transition of LTE₄ 440.2→301.2 m/z and LTE₄-d3 443.2→304.2 m/z was measured as described by Kishi et al (2001) or Hardy et al (2005). Both transitions were monitored for 500 ms resulting in 1 scan/sec. LTE₄ and LTE₄-d3 were monitored for quantitation by extracting ion chromatograms for the transitions 440.2→301.2 m/z and 443.2→304.2 m/z using Mass Hunter Quantitative Analysis Software (Agilent Technologies, Palo Alto, Calif.). In some experiments the transitions of LTE₄ 440.2→189.2 m/z and LTE₄-d3 443.2→192.2 m/z were monitored as qualifiers. This is described in detail below.

The column effluent from the analytical column flowed into the mass spectrometer. (The effluent could also be directed to waste.) The column effluent was directed to the MS only when LTE₄ was eluting from the analytical column. Upon entry into the MS, molecules were ionized or charged. Acetic acid was used as the ionizing agent. Following ionization, the LTE₄ molecules moved into the mass analyzers/filters.

As shown in FIG. 2, Multiple Reaction Monitoring (MRM) was used to quantitate molecules as they eluted from the column and were ionized. The triple quadrupole (QQQ) instrument was composed of 3 mass analyzers/filters (the quadrupoles). MRM was used to specifically filter for a molecule of interest in the first quadrupole, fragment that molecule in the second quadrupole, and then filter for only a single resulting fragment peak in the third quadrupole. Because a labeled analog of the molecule of interest will undergo identical fragmentation (i.e. MRM transitions), the peak area of the labeled molecule can be directly compared to the peak area of the endogenous, unlabeled molecule, providing quantitative information.

Intact LTE₄ molecules were ionized with acetic acid to form positively charged molecules with m/z 440.2. (The LTE₄ molecules could also be ionized to form negatively charged molecules with m/z 438.2, and the instrument could be operated in positive ion mode to analyze positively charged molecules, or in negative ion mode to analyze negatively charged molecules). Positively charged masses with m/z approximately 440.2 Da passed through the first quadrupole and all others were filtered out. The second quadrupole was used to fragment the LTE₄ ions. The third quadrupole was used to filter for ions of interest and send them to the detector. LTE₄ fragmentation produced ions with m/z ratio of 301.2 and 189.2. In one scan, the fragment with m/z 301.2 was selected. In another scan, the fragment with m/z 189.2 was selected. In yet another scan, labeled LTE₄ (i.e. LTE4-d3) with m/z 443.2 was selected, fragmented, and its fragments (304.2 m/z and 192.2 m/z) were detected. Several scans were averaged and at each scan the intensity of the ions was measured. The intensity of the ions over a range of scans was plotted as an “extracted ion chromatogram” (EIC). Following the MS analysis the area of the EIC for both the labeled and unlabeled molecules was computed. Since the quantity of the labeled molecule (LTE₄-d3) was known, this information was used to extrapolate the quantity of the unlabeled endogenous LTE₄ molecule. A standard curve was generated using known quantities of LTE₄ over a broad range (5-2,000 pg/ml) to ensure the method was linear across this range, using linear regression or quadratic regression with a 1/x weighting. A known quantity of LTE4-d3 was also analyzed prior to every batch of samples analyzed and the amount validated to ensure precision of the method.

MS: Intraday variation was monitored by spiking in labeled LTE₄ at concentrations of pg/ml and analyzing samples in triplicate on the same day. Interday variation was evaluated over 3 days. Relative error was calculated as [(found concentration)−(theoretical concentration)/spiked concentration]×100(%) as described by Kishi et al (2001). Linearity was determined using linear regression analysis as described by Westcott et al (1997).

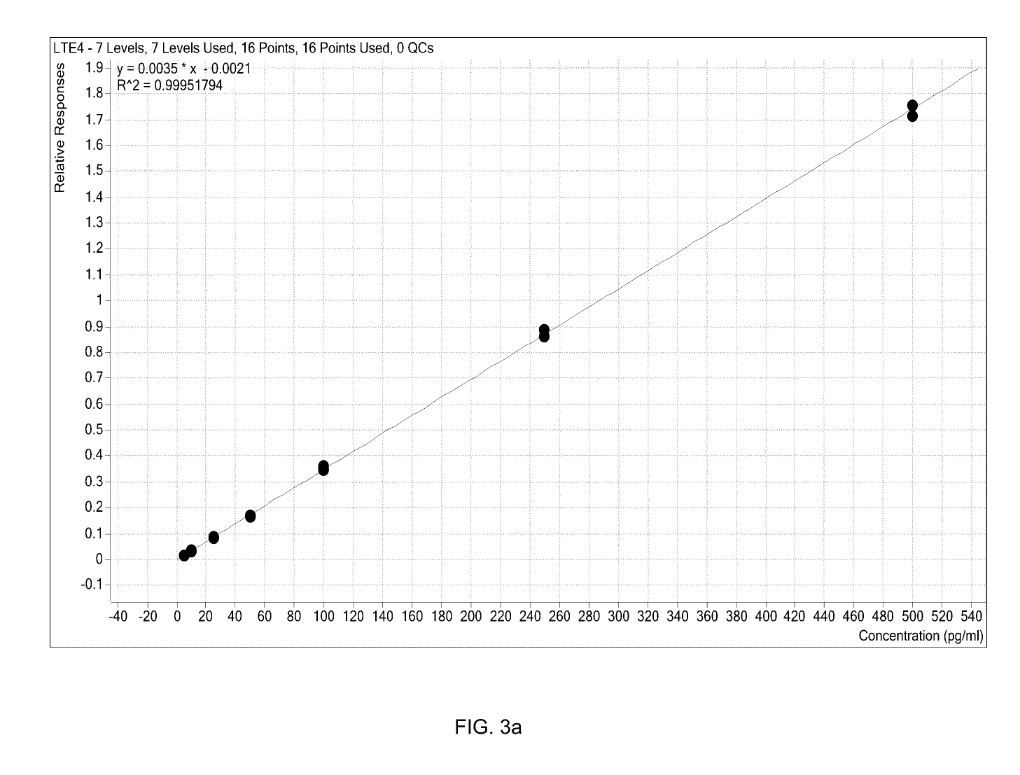

As shown in FIG. 2, it was possible to detect an LTE₄ standard using this method and calibration curves (5-1,000 pg/ml) with R² values of approximately 0.999 were routinely generated (FIG. 3a and FIG. 3b). Typical response values (i.e. Intensities) ranged from 300 for 5 pg/ml samples to 30,000 for 500 pg/ml samples while blank samples, composed of normal urine had responses under 20 (data not shown). Response values are arbitrary and have no units of measurement.

Standards from two independent sources were compared with the standard from Cayman Chemical (Ann Arbor, Mich.) producing a lower response than the one from Biomol (Plymouth Meeting, Pa.).

To assess the accuracy and precision of the present method over the physiological range, intra- and inter-day analyses were conducted over a three-day period using both standards and spiked urine. With the exception of two out of thirty-three measurements, percent CV's [CV=Coefficient of Variance] were under 10% and the average CV was 3.47% for all intra-day experiments. Similarly, the average CV for inter-day measurements was 6.55%. Recoveries ranged from 90.56 to 121.26% for the intra-day experiments, with an average of 100.56% for both intra- and inter-day experiments. Recoveries ranged from 94.75 to 106.58 in the inter-day experiments. This data is summarized in Table 2. Values for spiked urine samples are also given in Table 2 whereby urine was spiked with 0, 25, 100, or 250 pg/ml of LTE₄; values are listed as +0, +25, +100, +250. Intra- and Inter-day values are used to measure the precision and accuracy of an assay; LTE4 levels were measured once per day for 3 days for intra-day values, and three times in a single day for inter-day values.

TABLE 2

| | Intra-day Accuracy and Precision | | | | | | | | | Inter-day Accuracy and | | |
|---|---|---|---|---|---|---|---|---|---|---|---|---|
| | Day 1 | | | Day 2 | | | Day 3 | | | Precision (n = 3) | | |
| | AVG | CV | % R | AVG | CV | % R | AVG | CV | % R | AVG | CV | % R |
| Calibration stds (n = 2) | | | | | | | | | | | | |
| (pg/mL) | | | | | | | | | | | | |
| 5 | 4.63 | 17.17 | 92.58 | 6.06 | 2.28 | 121.26 | 5.30 | 3.06 | 105.90 | 5.33 | 13.47 | 106.58 |
| 10 | 10.51 | 1.02 | 105.15 | 9.18 | 7.55 | 91.82 | 10.30 | 11.27 | 102.97 | 10.00 | 7.15 | 99.98 |
| 25 | 22.64 | 6.53 | 90.56 | 23.17 | 2.63 | 92.67 | 26.50 | 3.42 | 106.02 | 24.10 | 8.69 | 96.42 |
| 50 | 50.18 | 0.83 | 100.35 | 43.78 | 4.35 | 87.56 | 48.18 | 1.25 | 96.35 | 47.38 | 6.91 | 94.75 |
| 100 | 106.93 | 0.05 | 106.93 | 102.14 | 2.25 | 102.14 | 98.48 | 0.85 | 98.48 | 102.52 | 4.13 | 102.52 |
| 250 | 264.05 | 3.00 | 105.62 | 267.04 | 1.08 | 106.81 | 254.53 | 0.61 | 101.81 | 261.87 | 2.49 | 104.75 |
| 500 | 477.81 | 0.76 | 95.56 | 488.63 | 0.13 | 97.73 | 496.99 | 1.58 | 99.40 | 487.81 | 1.97 | 97.56 |
| Spiked Urine (n = 5) | | | | | | | | | | | | |
| (pg/mL) | | | | | | | | | | | | |
| +0 | 60.54 | 5.91 | NA | 51.67 | 4.77 | NA | 63.49 | 2.23 | NA | 58.57 | 10.51 | NC |
| +25 | 83.42 | 1.74 | 91.51 | 77.19 | 4.34 | 102.08 | 91.42 | 3.72 | 111.71 | 84.01 | 8.49 | 101.77 |
| +100 | 163.75 | 3.88 | 103.21 | 152.82 | 2.28 | 101.15 | 160.43 | 4.80 | 96.94 | 159.00 | 3.52 | 100.43 |
| +250 | 328.36 | 2.32 | 107.13 | 302.54 | 4.24 | 100.35 | 329.14 | 2.73 | 106.26 | 320.01 | 4.73 | 104.58 |
| Continuing cal (100 pg/mL) | 103.24 | 0.42 | 103.24 | 93.02 | 0.52 | 93.02 | 94.08 | 1.35 | 94.08 | 96.78 | 5.81 | 96.78 |

### Example 2

This example illustrates that the method of the present invention for measuring LTE₄ is more precise and shows less variance than ELISA method for measuring LTE₄.

ELISA assays performed in-house were conducted using unprocessed and processed urine. Processing methods included using an affinity sorbent, an affinity purification column, solid phase extraction, and on-line HPLC. Urine samples were also sent to Cayman Chemical company for analysis, which is conducted using an affinity sorbent followed by ELISA for LTE₄.

Urine was obtained from 10 asthmatic subjects and LTE₄ levels were compared using ELISA and LCMSMS method. One aliquot was analyzed using LCMSMS on the day of collection. Remaining sample was aliquoted and stored at −80° C. Frozen aliquots were either sent for ELISA analysis or were analyzed using LCMSMS. Calibration curves were run prior to LCMSMS (R2=0.9995, FIG. 2). Creatinine values were obtained through Cayman. No R2 values were provided with the ELISA results.

As shown in Table 3, following normalization to creatinine, the ELISA resulted in values ranging from 639 to 5685 pg/ml with CV's ranging from 2.60% to 26.42% (average 14.34%). The present method resulted in values ranging from 29 to 143 pg/ml with CV's ranging from 0.44% to 7.36% (average 2.99%).

TABLE 3

| | | Final | | Norm. | | |
|---|---|---|---|---|---|---|
| Sample | Method | Conc. | Creatinine | LTE4 | AVG | CV |
| 1 | LCMS | 135.55 | 159.3840 | 85.0438 | 84.13 | 1.54 |
| | LCMS | 129.91 | 159.3840 | 81.5087 | 82.02 | 0.87 |
| | ELISA | 1814.66 | 159.3840 | 1138.5459 | | 23.42 |
| 2 | LCMS | 107.13 | 170.6610 | 62.7712 | 61.07 | 3.95 |
| | LCMS | 90.36 | 170.6610 | 52.9448 | 53.82 | 2.30 |
| | ELISA | 1502.72 | 170.6610 | 880.5292 | | 26.42 |
| 3 | LCMS | 38.97 | 116.0370 | 33.5805 | 33.45 | 0.53 |
| | LCMS | 34.98 | 116.0370 | 30.1486 | 29.42 | 3.50 |
| | ELISA | 3515.52 | 116.0370 | 3029.6543 | | 10.41 |
| 4 | LCMS | 118.87 | 96.5180 | 123.1604 | 127.77 | 5.10 |
| | LCMS | 110.84 | 96.5180 | 114.8411 | 114.00 | 1.05 |
| | ELISA | 617.67 | 96.5180 | 639.9532 | | 10.48 |
| 5 | LCMS | 69.99 | 67.4579 | 103.7480 | 106.62 | 3.81 |
| | LCMS | 62.80 | 67.4579 | 93.0946 | 98.02 | 7.11 |
| | ELISA | 1022.55 | 67.4579 | 1515.8343 | | 14.68 |
| 6 | LCMS | 82.23 | 58.5341 | 140.4863 | 141.95 | 1.45 |
| | LCMS | 84.17 | 58.5341 | 143.7951 | 143.30 | 0.49 |
| | ELISA | 1120.93 | 58.5341 | 1915.0034 | | 13.33 |
| 7 | LCMS | 52.10 | 67.5873 | 77.0926 | 78.87 | 3.18 |
| | LCMS | 51.39 | 67.5873 | 76.0312 | 72.27 | 7.36 |
| | ELISA | 652.74 | 67.5873 | 965.7732 | | 2.60 |
| 8 | LCMS | 81.98 | 95.9391 | 85.4551 | 87.86 | 3.87 |
| | LCMS | 83.32 | 95.9391 | 86.8457 | 87.54 | 1.13 |
| | ELISA | 5627.56 | 95.9391 | 5865.7628 | | 8.74 |
| 9 | LCMS | 111.99 | 105.0380 | 106.6177 | 105.23 | 1.86 |
| | LCMS | 110.44 | 105.0380 | 105.1451 | 105.47 | 0.44 |
| | ELISA | 1102.95 | 105.0380 | 1050.0486 | | 23.47 |
| 10 | LCMS | 133.07 | 189.5490 | 70.2053 | 67.52 | 5.63 |
| | LCMS | 128.12 | 189.5490 | 67.5937 | 69.86 | 4.59 |
| | ELISA | 2100.39 | 189.5490 | 1108.0987 | | 9.82 |

A comparison of ELISA and the present LCMSMS method shows that the present method is superior to the ELISA method. The ELISA method led to higher estimations of LTE₄. In the present invention, the ELISA assay was conducted at an off-site laboratory according to their protocol and no affinity-purification was used. However, samples analyzed via ELISA using various affinity purification methods also showed similarly high values and high % CV (data not shown). These findings are consistent with others' assessment of LTE₄ ELISA assays. A possible explanation for the discrepancy is interference from contaminants for the antibody binding sites during the LTE₄ ELISA. For ELISA assays that incorporate purification, although somewhat lower values are reported, high variance can still confound data analysis. Conversely, the lower variance reported using the present methods enable clinical investigators to better differentiate between patient phenotypes.

### Example 3

This example illustrates the integrity of LTE₄ samples as measured using the method of the present invention. These results can used to determine shipping and storage of urine samples for the purpose of LTE₄ analysis.

Sample integrity was measured using the method of the present invention following various storage procedures: samples analyzed on the day of collection, following 24 hours at 30° C. following a single freeze/thaw cycle, following 24 hours at room temperature following a single freeze/thaw cycle, following a single freeze/thaw cycle, following up to 7 days at 4° C. without freezing and thawing, and following five freeze/thaw cycles. As shown in Table 4, storing the samples at 4° C. for 7 days had the least affect while samples stored at 30° C. for 24 h underwent the most loss of LTE₄. Perhaps surprisingly, single and multiple (5) freeze/thaw cycles had an equivalent effect on samples, with losses of 11.43% and 13.16% respectively. These results may be used to determine shipping and storage of urine samples for the purpose of LTE₄ analysis.

TABLE 4

| | | | 30 C. for 24 hrs > | | RT for 24 hrs > | | | | | | | | |
|---|---|---|---|---|---|---|---|---|---|---|---|---|---|
| | Day of | | Storage @ −80 C. > | | Storage @ −80 C. > | | Storage @ −80 C. > | | 4 C. for 7 days | | | | |
| Sample | collection | | 1 freeze/thaw | | 1 freeze/thaw | | 1 freeze/thaw | | w/o freezing | | Multiple Freeze/Thaws | | |
| No. | AVG | CV | AVG | CV | AVG | CV | AVG | CV | AVG | CV | AVG | CV | % D from fresh |
| 1 | 73.50 | 0.28 | 58.13 | 0.04 | 53.30 | 4.15 | 58.39 | 1.20 | 67.43 | 8.96 | 59.32 | 3.51 | −18.30 |
| 2 | 71.22 | 0.91 | 55.89 | 5.26 | 55.53 | 4.16 | 62.37 | 6.97 | 65.02 | 1.67 | 62.77 | 7.19 | −12.69 |
| 3 | 71.94 | 2.90 | 53.76 | 1.93 | 54.56 | 2.89 | 61.18 | 2.25 | 65.37 | 8.10 | 60.51 | 7.57 | −16.33 |
| 4 | 74.25 | 3.33 | 52.66 | 1.58 | 55.69 | 2.13 | 62.76 | 5.29 | 59.31 | 0.75 | 65.98 | 5.27 | −7.71 |
| 5 | 65.45 | 4.21 | 53.32 | 1.11 | 56.66 | 2.17 | 60.49 | 0.75 | 60.68 | 0.24 | 63.97 | 6.20 | −10.79 |
| Average | 71.27 | 2.33 | 64.75 | 1.99 | 55.15 | 3.10 | 61.04 | 3.29 | 63.56 | 3.94 | 62.51 | 5.95 | −13.16 |
| Sdev | 4.87 | | 4.10 | | 2.31 | | 2.85 | | 5.38 | | | | |

| | | | | |
|---|---|---|---|---|
| % D from fresh | −26.21 | −25.51 | −15.47 | −11.43 |

Because initial steps can vary greatly between laboratories, sample handling studies also included a comparison of centrifuged samples versus non-centrifuged samples and also the addition of protease inhibitors. Results are shown in Table 5. In general there was no significant difference between samples regardless of the time of centrifugation. Similarly, the addition of protease inhibitors in clinical samples had no effect on the recovery (data not shown).

TABLE 5

| Std (p-value) | Spin | AVG | CV | % R |
|---|---|---|---|---|
| 0 pg (0.46) | Y | 21.72 | 10.1 | NC |
| | N | 22.85 | 4.33 | NC |
| 25 pg (0.05) | Y | 40.72 | 1.46 | 76.0172 |
| | N | 45.41 | 6.15 | 90.24 |
| 100 pg (0.57) | Y | 108.93 | 1.32 | 87.2053 |
| | N | 111.90 | 7.36 | 89.04 |
| 250 pg (0.21) | Y | 253.89 | 3.73 | 92.8677 |
| | N | 265.20 | 3.45 | 96.94 |

### Example 4

This example illustrates the determination of clinically relevant cut-off values of LTE₄ for diagnosis of asthma.

The asthma group (N=44) consisted of students aged 6 to 15 at the Kunsberg School for children with chronic respiratory diseases located on the campus of National Jewish Health. All children in this cohort had physician-diagnosed asthma. This group was compared to 38 non-asthmatic children of similar age from an elementary school in the Denver area. Spot urine samples for the asthmatic group were collected in the fall of 2007 and kept at minus 70 degree Celsius before assay. Fractional exhaled nitric oxide (FENO) was measured at the same time as spot urine collection using a NIOX (Aerocrine A B, Solna, Sweden) FENO analyzer at standard settings. Collection of spot urines from healthy control non-asthmatic children was performed in the fall of 2008 but FENO was not measured in this group. Information about asthma, food allergy eczema or hay fever was elicited from all families at the time of recruitment. Families in the asthma group also reported on current asthma medication usage and asthma severity over the previous year. Ethical and scientific approval was obtained from the National Jewish Institutional Review Board.

Although 37 of the 44 Kunsberg schoolchildren (84%) were receiving daily inhaled corticosteroids (ICS) as prescribed by their providers, most of them continued to have persistent disease. Eighteen (41%) complained of nighttime symptoms once a week or less, 20 (45%) 2 to 4 times per week and 6 (14%) 5 to 7 days per week and 7 (16%) required hospitalization in the previous year. Thirty-seven asthmatic children (84%) and ten children in the control group (26%) reported some atopic history.

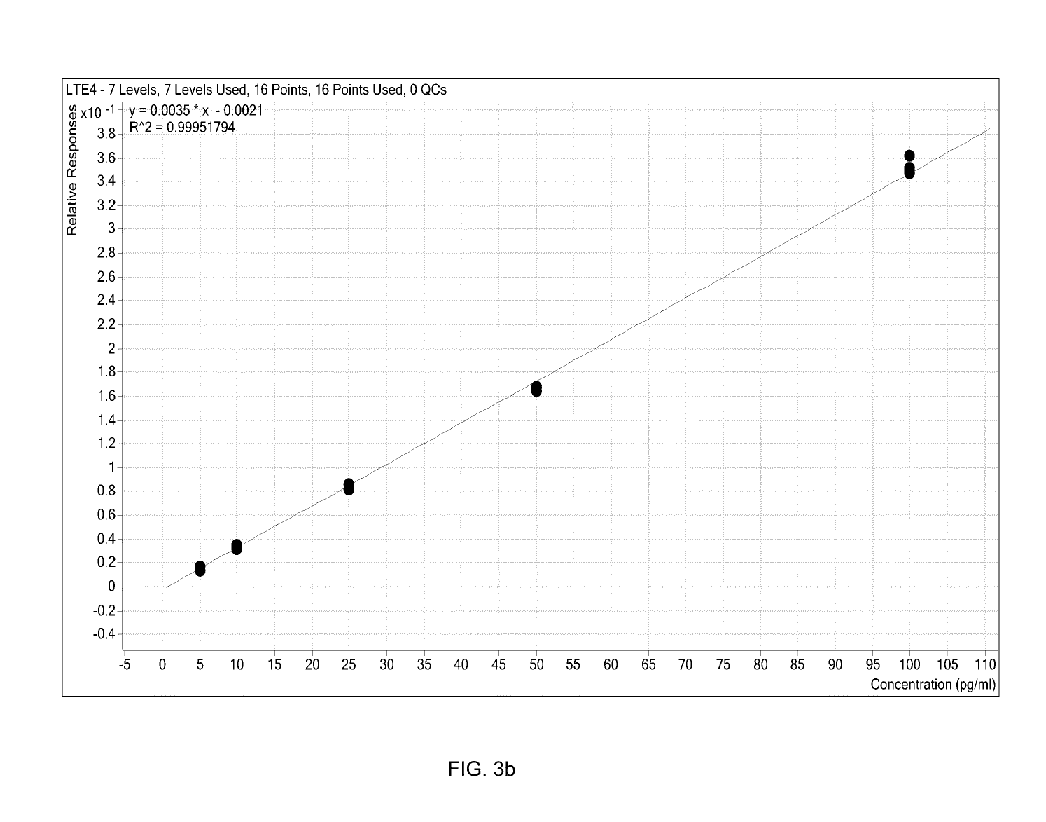

The LTE₄ levels were measured using the method described in Example 1. The median LTE₄ level in the asthma group was 64.4 picograms per milligram (pg/mg) creatinine (25^{th} and 75^{th} percentiles 47 and 109.4 pg/mg, range 13.2 to 311.4 pg/mg). In the control group, median LTE₄ levels measured 46.7 pg/mg (25^{th} and 75^{th} percentiles 31.2 and 62.5 pg/mg, range 7.0 to 115.1 pg/mg). Median FENO levels measured 17.1 parts per billion (ppb) (25^{th} and 75^{th} percentiles 10.7 to 37.5 ppb, range 4.5 to 143.7 ppb). LTE₄ levels were significantly (p=0.002, unpaired t-test) higher in the asthmatic children (geometric (geo) mean: 4.2 pg/mg creatinine, 95^{th} CI: 4.01-4.38) compared to the non-asthmatic controls (geo mean 3.76 pg/mg, 95^{th} CI: 3.56-3.96) (FIG. 4A).

FIG. 4B illustrates the receiver operator characteristic (ROC) for the relationship between LTE₄ and asthma diagnosis (area under the curve (AUC)=0.69, p=0.005). An LTE₄ value at or above 53 pg/mg yielded the highest accuracy with a sensitivity of 70.4% and specificity of 63.2%, a positive likelihood ratio (LR) of 1.91 and a negative LR of 0.47. A value at or above 112.6 pg/mg yielded a sensitivity of 25% (11 out of 44 asthmatics), a specificity of 97.4% (1 out of 38 non-asthmatics) and a positive LR of 9.5. The high positive likelihood ratio at the cut-off point of 112.6 pg/mg suggests that LTE₄ may be a valuable diagnostic test in the tertiary clinic setting with a similar sensitivity/specificity profile as 20% diurnal variability in peak flow or 12% reversibility in forced expiratory volume in 1 second (FEV₁) (Goldstein 2001). As an example, assuming a high asthma prevalence (pre-test probability) in a tertiary referral clinic of 70%, if a child's LTE₄ value was determined to be at or above 112.6 pg/mg, the post-test probability would be 96% (although a negative test would not appreciably change disease prevalence). As the test is effort independent, its value as a diagnostic biomarker may be greatest in the youngest children when diagnosing asthma is particularly challenging due to the unavailability of pulmonary function tests or FENO and phenotypic overlap with “wheezy bronchitis”.

Aside from their diagnostic usage, biomarkers are also valuable in asthma management. For example, the utilization of FENO measurements has been studied as a guide towards initiation of stepped up ICS therapy to prevent exacerbation and hospitalization in children (Szefler 2008). In the present study, FENO levels were associated with hospitalizations but not with daily symptoms. The most accurate cutoff point for FENO as an indicator of hospitalization in the past year was 23 ppb (AUC=0.81, p=0.01). Six out of 7 (86% sensitivity) recently hospitalized children had FENO levels at or above this level and 26 out of 37 (70% specificity) children who were not hospitalized had values below this level (p=0.009, 2-tailed Fisher's exact test).

For LTE₄, the most accurate cutoff point to identify children who were hospitalized was at or above 99.6 pg/mg (AUC 0.70, p=0.30). Five out of 7 (71% sensitivity) children requiring hospitalization had an LTE₄ level at or above this value and 30 out of 37 (81% specificity) children not requiring hospitalization had levels below this cutoff point (p=0.01). As such, a positive LTE₄ test has a greater predictive value in identifying hospitalized children than does a positive FENO test (positive LR 3.74 for LTE₄, 2.87 for FENO) (Table 6). In our cohort, for example where the prevalence of hospitalization was 16%, the post-test prevalence would be 42% in children with a positive LTE₄ test and 35% for those with a positive FENO test. In contrast, a negative FENO test appears to have greater value in identifying non-hospitalized children (negative LR=0.35 for LTE₄, 0.20 for FENO). In the context of this study, for example, where 84% of the children were not hospitalized in the past year, the post-test prevalence of non-hospitalized children would be 96% given a negative FENO test and 94% given a negative LTE₄ test. Using a parallel approach measuring both biomarkers produces higher sensitivity than does either biomarker alone. All 7 (100% sensitivity) of the children with recent hospitalizations either had an FENO at or above 23 ppb or an LTE₄ level at or above 99.6 pg/mg while 25 out of 37 (68% specificity) non-hospitalized children had FENOs and LTE₄ levels below these cut-off points (positive LR=3.03, negative LR=0, p=0.001).

TABLE 6

| | Hospitalized | Post-test prevalence | Not hospitalized | Post-test prevalence |
|---|---|---|---|---|
| | (n = 7) | of hospitalization | (n = 37) | of no hospitalization |
| | n, (sensitivity), | given positive test | n, (specificity), | given negative test |
| Biomarker | Likelihood | (assume 16% pre- | Likelihood | (assume 84% pre- |
| profile | Ratio (LR) | test prevalence) | Ratio (LR) | test prevalence) |
| LTE₄ | 5 (71%) | 42% | 30 (81%) | 94% |
| (=/>99.6 | Positive LR | | Negative LR | |
| mg/pg) | 3.74 | | 0.36 | |
| FENO | 6 (86%) | 35% | 26 (70%) | 96% |
| (=/>23 ppb) | Positive LR | | Negative LR | |
| | 2.87 | | 0.20 | |
| FENO | 7 (100%) | 37% | 25 (67%) | 100% |
| (=/>23 ppb) | Positive LR | | Negative LR | |
| or LTE₄ | 3.03 | | 0 | |
| (=/>99.6 | | | | |
| pg/mg) | | | | |

### Example 5

This example illustrates the determination of clinically relevant cut-off values of LTE₄ for prediction of exacerbation of asthma.

One of the major challenges in adult and childhood asthma is how to predict disease worsening so as to preempt severe asthma exacerbations. Reliance on symptom patterns or peak flow monitoring does not appear to provide sufficient warning of upcoming exacerbation (Covar 2008) perhaps because many patients with chronic asthma have poor symptom recognition while changes in peak flows are effort dependant and may be insensitive until later in the exacerbation process. As such, it has been suggested that measurement of biological markers of airway inflammation may provide a sensitive indicator of airway changes that occur at the very beginning of this process. For example, the utilization of fractional exhaled nitric oxide (FENO) measurements has been studied as an additional indicator with conventional asthma monitoring for calibrating inhaled corticosteroid (ICS) therapy to prevent exacerbations and maintain the lowest possible maintenance dose (Szefler 2008, Karitonov 1996, Doucet 1990). Together, these studies suggest that while adding FENO monitoring to asthma guidelines may be a useful strategy to safely guide ICS reduction in controlled asthmatics, it is not effective in signaling the need for stepped-up ICS therapy to prevent exacerbations and in some cases, may lead to higher ICS usage without significant health benefits. As such, the value of a negative FENO test (i.e. the negative likelihood ratio (LR)) for guiding reductions in controller therapy appears to be greater than the value of a positive FENO test in predicting the need for stepped-up therapy (positive LR). In this context, the use of additional biomarkers with greater positive LR values may be crucial in calibrating the balance between ICS reduction vs additional therapy. As levels are largely unaffected by ICS usage (Manso 1992, O'Shaughnessy 1993), urine LTE₄ may be an attractive choice for disease monitoring in this context. However, prior to the present invention, there were no studies available that determined relevant cut-off points allowing a clinician to utilize this test to monitor asthma severity. As described in detail in this Example, the present inventors, using the methodologies of the present invention, determined clinically relevant cut-off values for LTE₄ that may be used to predict asthma exacerbations.

Children (N=44) with physician diagnosed asthma aged 6 to 15 at the Kunsberg School located on the campus of National Jewish Health were followed for a 5.5 month period (Dec. 3, 2007 through Apr. 17, 2008). Up to 8 urine samples were collected on consecutive schooldays during the first half of the study. Urine was collected at approximately the same time each day (11 AM to 1 PM), frozen at −70° C. and than batch assayed for the level of LTE₄ using the LCMSMS method of the present invention. Fractional exhaled nitric oxide (FENO) was measured at the same time as urine collection using a NIOX (Aerocrine AB, Solna, Sweden) FENO analyzer at standard settings. Once during the study period, children performed pre- and post albuterol (4 puffs) spirometry on an office spirometer after discontinuing short (4 hours previously) and long-acting (24 hours previously) bronchodilator treatment. On a weekly basis, children were asked about the incidence of any asthma emergency room (ER) urgent care (UC) visits for asthma; such information was crosschecked with parents as well. An additional source of information was from school nurses provided information about ER and UC visits as well as the initiation of prednisone bursts. Ethical and scientific approval for this study was obtained from the National Jewish Institutional Review Board.

The outcome measure was presence or absence of an asthma exacerbation for an individual child during the 6-month study period. This outcome measure was determined in 2 ways. The primary exacerbation outcome was presence of an urgent care or ER visit during the study period. As a sensitivity analysis, this outcome was restricted to those children who were required a prednisone burst after an ER or UC visit. For the biomarker predictors, mean LTE₄ and FENO levels were calculated based on the repeated measurements from the 1^{st} 2.5 months of the study. Pre-bronchodilator percent-predicted forced expiratory value in 1-second (ppFEV₁), FEV₁ to forced expiratory value ratio (FEV₁/FVC) and FEV₁ reversibility (reFEV₁) were measured on 1 occasion for each child the 5 months of the study. Receiver operator curves (ROC) were constructed for the predictors with a child's exacerbation status (1 or more ER/UC visit or 1 or more prednisone burst) as the outcome. The predictor levels with the highest accuracy (true positive+true negatives divided by false positives+false negatives) were used as “cut-off” point for categorical groupings and the association between predictors and exacerbation was assessed using a 2-tailed Fisher's exact test. Positive likelihood ratios were calculated as sensitivity divided by (1−specificity) and negative likelihood ratios were calculated as (1−sensitivity) divided by specificity at the respective cut-off points. Post-test odds were calculated (pre-test odds×likelihood ratio) and then transformed to post-test prevalence (PTP). Statistical analyses were performed using JMP software (SAS, NC).

Thirty-seven of 44 children in the asthma group (84%) were receiving daily inhaled corticosteroids (ICS) as prescribed by their providers. Most of the children had moderate or severe asthma despite use of controller medications. Eighteen (41%) complained of nighttime symptoms once a week or less, 20 (45%) 2 to 4 times per week and 6 (14%) 5 to 7 days per week.

The median LTE₄ level was 75.5 picograms per milligram (pg/mg) creatinine (25^{th} and 75^{th} percentiles 52.8 and 105.3 pg/mg, range 12.5 to 295.4 pg/mg). Median FENO levels measured 16.3 parts per billion (ppb) (25^{th} and 75^{th} percentiles 9.4 to 34.8 ppb, range 5.4 to 93.7 ppb). Median ppFEV1 was 97.5 (range 61 to 130%), median FEV1 to FVC ration was 81.5, range 52 to 99% and median reversibility was 10% (range −6 to 27). Twelve of the 44 (27%) children had at least one urgent care or emergency room visit (9 ER, 3 UC visits) of which 9 required a prednisone burst.

The most accurate cut off point with their respective sensitivities and specificities and p-values for each predictor variable are summarized in Table 7. For LTE₄, the most accurate cutoff point was at or above 106.4 pg/mg for both UC and ER visits (AUC 0.70, p=0.12) and for prednisone requirement (AUC 0.72, p=0.09). Seven out of 12 (58% sensitivity) children requiring ER or urgent care visits and 4 out of 32 (88% specificity) children not requiring an ER or UC visit had mean LTE₄ levels at or above 106.4 pg/mg (p=0.004). Five out of 9 (55% sensitivity) children requiring prednisone and 6 of 35 (83% specificity) not requiring prednisone had levels at or above 106.4 pg/mg. The most accurate cutoff point for FENO as an indicator of UC or ER visit was 13.6 ppb (AUC=0.66, p=0.22) and for prednisone requirement it was 16.5 ppb (AUC=0.57, p=0.22). Eleven out of 12 (92% sensitivity) children with exacerbations and 18 out of 32 children without exacerbations (specificity 44%) had FENO levels at or above 13.6 ppb (p=0.04). Seven of 9 (78% sensitivity) children requiring prednisone and 20 of 35 (43% specificity) not requiring prednisone bursts had FENO levels above 16.5 ppb. The optimal cut-off for ppFEV₁ was below 91% for both ER/UC visits (AUC 0.70, p=0.04) and need for prednisone (AUC 0.68, p=0.08). Eight out of 12 children with ER/UC visits (67% sensitivity) and 11 out of 32 (66% specificity) without ER/UC visits had ppFEV₁ values below 91% (p=0.09). Six of the 9 children requiring prednisone (67% sensitivity) and 13 of the 35 children (63% specificity) not requiring prednisone had ppFEV₁ values below 91%.

Table 7 further summarizes the positive and negative LRs and PTPs for each predictor. The highest positive likelihood ratio was achieved with LTE₄ levels with FENO yielding the best negative likelihood ratio and ppFEV1 achieving intermediate positive and negative likelihood ratios. As such the most predictive positive test for exacerbation would be LTE₄ at or above 106.4 pg/mg with less prediction in lower values. In contrast, negative FENO values may be more predictive than positive values.

TABLE 7

| Biomarker | LTE₄ (=/>106.4 | FENO (=/>13.6 or | |
|---|---|---|---|
| Profile | mg/pg) | 16.1 ppb) | FEV₁ (<91%) |
| AUC: | 0.70: UC/ER | 0.66: UC/ER Visits | 0.70: UC/ER Visits |
| | Visits | | |
| | 0.72: Prednisone | 0.56: Prednisone | 0.68: Prednisone |
| | Bursts | Bursts | Bursts |
| Sensitivity | 58%: UC/ER | 92%: UC/ER Visits | 67%: UC/ER Visits |
| | Visits | 78%: Prednisone | 67%: Prednisone |
| | 55%: Prednisone | Bursts | Bursts |
| | Bursts | | |
| Specificity | 88% UC/ER | 44%: UC/ER Visits | 66%: UC/ER |
| | 83% Prednisone | 43%: Prednisone | Visits |
| | Bursts | Bursts | 63% Prednisone |
| | | | Bursts |
| Positive LR | 4.83: UC/ER | 1.39: UC/ER Visits | 2.03: UC/ER visits |
| Negative LR | 4.23: Prednisone | 1.16: Prednisone | 1.81: Prednisone |
| | 0.48: ER/UC | 0.18: ER/UC Visits | 0.5: ER/UC visits |
| | 0.54: Prednisone | 0.51: Prednisone | 0.52: Prednisone |
| Post-test | 64%: ER/UC Visits | 34% ER/UC Visits | 43%: ER/UC Visits |
| Prevalence for | 51% Prednisone | 22% Prednisone | 31% Prednisone |
| Positive Test | Bursts | Bursts | Bursts |
| Assume 27% | | | |
| prevalence for | | | |
| ER/UC visits and | | | |
| 20% prevalence for | | | |
| prednisone bursts | | | |
| Post-Test | 85% no ER/UC | 94% no ER/UC visit | 84% no ER/UC |
| Prevalence for | visit | 89% no Prednisone | visit |
| Negative Test | 88% no | Burst | 88% no |
| Assume 73% | Prednisone Burst | | Prednisone Burst |
| prevalence for no | | | |
| ER/UC visits and | | | |
| 80% prevalence for | | | |
| no prednisone | | | |
| bursts | | | |

* Assuming sensitivities of 99% for this analysis

### REFERENCES

Cai C, Yang J, Hu S, Zhou M, Guo W. Relationship between urinary cysteinyl leukotriene E4 levels and clinical response to antileukotriene treatment in patients with asthma. Lung 2007; 185:105-12.
Hardy G, Boizel R, Bessard J, Cracowski J L, Bessard G, Halimi S, Stanke-Labesque F. Urinary leukotriene E4 excretion is increased in type 1 diabetic patients: a quantification by liquid chromatography-tandem mass spectrometry. Prostaglandins Other Lipid Mediat. 2005 December; 78(1-4):291-9.
Hevko, J M, and Murphy R C. (2001). Electrospray ionization and tandem mass spectrometry of cysteinyl eicosanoids; Leukotriene C4 and FOG7. JASMS 12: 763-771
Kishi N, Mano N, Asakawa N. Direct injection method for quantitation of endogenous leukotriene E4 in human urine by liquid chromatography/electrospray ionization tandem mass spectrometry with a column-switching technique. Anal Sci. 2001 June; 17(6):709-13.
Kumlin M, Stensvad F, Larsson L, Dahlen B, Dahlen SE. Validation and application of a new simple strategy for measurements of urinary leukotriene E4 in humans. Clin Exp Allergy. 1995 May; 25(5):467-79.
Mai X M, Bottcher M F, Bruhammar M, Nilsson L, Zetterstrom O. Urinary inflammatory mediators and inhalation of hypertonic saline in children. Allergy. 2005 January; 60(1):60-4.
Malmstrom K, Rodriguez-Gomez G, Guerra J, Villaran C, Pineiro A, Wei L X. Oral montelukast, inhaled beclomethasone, and placebo for chronic asthma. A randomized, controlled trial. Montelukast/Beclomethasone Study Group. Ann Intern Med 1999; 130:487-95.
Rabinovitch N, Zhang L, Gelfand E W. Urine leukotriene E4 levels are associated with decreased pulmonary function in children with persistent airway obstruction. J Allergy Clin Immunol 2006; 118:635-40.
Rabinovitch, N, Strand, M., Stuhlman, K., Gelfand, E W. Exposure to tobacoo smoke increases LTE₄-related albuterol usage and response to montelukast. J Alergy Clin Immunol 2008 121: 1365-1371
Szefler S J, Phillips B R, Martinez F D, Chinchilli V M, Lemanske R F, Strunk R C. Characterization of within-subject responses to fluticasone and montelukast in childhood asthma. J Allergy Clin Immunol 2005; 115:233-42.
Westcott J Y, Johnston K, Batt R A, Wenzel S E, Voelkel N F. Measurement of peptidoleukotrienes in biological fluids. J Appl Physiol. 1990 June; 68(6):2640-8.
   Zhang J, Yu C, Holgate S T, Reiss T F. Variability and lack of predictive ability of asthma end-points in clinical trials. Eur Respir J 2002; 20:1102-9.
Kumlin M. Measurement of leukotrienes in humans. Am J Respir Crit Care Med 2000; 161 (2): S102-S106.
Smith C M, Hawksworth R J, Thien F C, Christie P E, Lee T H. Urinary leukotriene E4 in bronchial asthma. Eur Respir J 1992 June; 5 (6): 693-9.
Westcott J Y, Maxey K M. Macdonald J, Wenzel S E. Immunoaffinity resin for purification of urinary leukotriene E4. Prostaglandins Other Lipid Mediat 1998; 55: 301-21.
Doucet M Y, Jones T R, Ford-Hutchinson A W. Responses of equine trachealis and lung parenchyma to methacholine, histamine, serotonin, prostanoids, and leukotrienes in vitro. Can J Physiol Pharmacol 1990; 68:379-83.
Gyllfors P, Kumiln M, Dahlén S E, Gaber F, Ehrs P O, Dahlen B. Relation between bronchial responsiveness to inhaled leukotriene D4 and markers of leukotriene biosynthesis. Thorax 2005 November; 60(11):902-8.
Busse W, Kraft M. Cysteinyl leukotrienes in allergic inflammation: strategic target for therapy. Chest 2005; 127:1312-26.
Armstrong M, Liu, A H, Harbeck R, Reisdorph R., Rabinovitch N, Reisdorph N. Leukotriene-E4 in human urine: Comparison of on-line purification and liquid chromatography-tandem mass spectrometry to affinity purification followed by enzyme immunoassay. J Chromatograph B. doi:10.1016/j.jchromb.2009.08.011.
Goldstein M F, Veza B A, Dunsky E H, Dvorin D J, Belecanech G A, Haralabatos I C. Comparisons of peak diurnal expiratory flow variation, postbronchodilator FEV (1) responses, and methacholine inhalation challenges in the evaluation of suspected asthma. Chest 2001 April; 119(4):1001-10.
Szefler S J, Mitchell H, Sorkness C A, Gergen P J, O'Connor G T, Morgan W J, Kattan M, Pongracic J A, Teach S J, Bloomberg G R, Eggleston P A, Gruchalla R S, Kercsmar C M, Liu A H, Wildfire J J, Curry M D, Busse W W: Management of asthma based on exhaled nitric oxide in addition to guideline-based treatment for inner-city adolescents and young adults: a randomised controlled trial. Lancet 2008 Sep. 20; 372(9643):1065-72.
Covar R A, Szefler S J, Zeiger R S, Sorkness C A, Moss M, Mauger D T, Boehmer S J, Strunk R C, Martinez F D, Taussig L M; Childhood Asthma Research and Education Network. Factors associated with asthma exacerbations during a long-term clinical trial of controller medications in children. J Allergy Clin Immunol 2008 October; 122(4): 741-747.
S A Kharitonov, D H Yates, P J Barnes. Inhaled glucocorticoids decrease nitric oxide in exhaled air of asthmatic patients. Am. J. Respir. Crit. Care Med 1996; 153: 454-457.
Manso G, Baker A J, Taylor I K, Fuller R W. In vivo and in vitro effects of glucocorticosteroids on arachidonic acid metabolism and monocyte function in nonasthmatic humans. Eur Respir J 1992; 5:712-6.
O'Shaughnessy K M, Wellings R, Gillies B, Fuller R W. Differential effects of fluticasone propionate on allergen-evoked bronchoconstriction and increased urinary leukotriene E4 excretion. Am Rev Respir Dis 1993; 147:1472-6.
Westcott J Y, Maxey K M. Macdonald J, Wenzel S E. Immunoaffinity resin for purification of urinary leukotriene E4. Prostaglandins Other Lipid Mediat 1998; 55: 301-21.

What is claimed is:

1. A method for determining the level of a leukotriene present in a sample from a donor, comprising the steps of:
a) subjecting the sample to high performance liquid chromatography (HPLC) to partially purify the sample, and
b) subjecting the partially purified sample to tandem mass spectrometry.

2. The method of claim 1, wherein the leukotriene is selected from the group consisting of LTA₄, LTB₄, LTC₄, LTD₄, LTE₄, and LTF₄.

3. The method of claim 1, wherein the HPLC system comprises an enrichment column and an analytical column, wherein the two columns are connected to each other by a switching valve, the analytical column is connected to a mass spectrometer, and the effluent from the analytical column is directed into the mass spectrometer as the leukotriene elutes from the analytical column.

4. The method of claim 3, wherein the mass spectrometer comprises more than one quadrupole; leukotriene molecules with a specific mass to charge ratio (“m/z ratio”) are selected in the first quadrupole of the mass spectrometer and are dissociated to form leukotriene fragments in the second quadrupole; and one leukotriene fragment is selected for quantitative analysis and another leukotriene fragment is used for qualitative analysis.

5. The method of claim 4, wherein the leukotriene is LTE₄ and wherein, the leukotriene molecules selected in the first quadrupole have a m/z ratio in positive mode of about 440.2 and a m/z ratio in negative mode of about 438.2.

6. The method of claim 5, wherein the LTE₄ fragments comprise fragments of m/z ratios of about 189.2, 205.2, 265.2, 283.2, and 301.2 Da in positive mode and about 235.2, 289.2, 317.2, 333.2, 351.2 and 420 Da in negative mode.

7. The method of claim 6, wherein in positive mode the LTE₄ fragment of m/z ratio of 301.2 is selected for quantitative analysis and the LTE₄ fragment of m/z ratio of 189.2 is selected for qualitative analysis.

8. The method of claim 6, wherein in negative mode the LTE₄ fragment of m/z ratio of 333.2 is selected for quantitative analysis and the LTE₄ fragment of m/z ratio of 351.2 is selected for qualitative analysis.

9. The method of claim 3, wherein the pH of the HPLC solvent is in the range of about 2.5 to about 8.5.

10. The method of claim 1, wherein a known amount of labeled leukotriene is added to the sample.

11. The method of claim 5, wherein the leukotriene is labeled with a stable isotope.

12. The method of claim 1, wherein prior to step a, the sample is centrifuged and the supernatant is subjected to step a.

13. The method of claim 1, wherein the sample may be urine, blood, saliva, sputum, broncho-alveolar fluid or exhaled breath condensate.

14. A method of determining the susceptibility of a donor to treatment with a leukotriene modifier,
comprising obtaining a sample from the donor and determining the level of leukotriene in the sample selected from the group consisting of LTA₄, LTB₄, LTC₄, LTD₄, LTE₄, and LTF₄, and combinations thereof,
wherein presence of the leukotriene in the sample at an elevated level as compared to a baseline level established from a control sample, identifies the donor as susceptible to treatment with the leukotriene modifier.

15. The method of claim 14, wherein the step of determining the level of leukotriene in the sample comprises determining the level of LTE₄.

16. The method of claim 14, wherein the leukotriene modifier is selected from the group consisting of a leukotriene receptor antagonist and a leukotriene synthesis inhibitor.

17. The method of claim 16, wherein the leukotriene receptor antagonist is selected from the group consisting of montelukast, zafirlukast and pranlukast.

18. The method of claim 16, wherein the leukotriene modifier is an inhibitor of the 5-lipoxygenase pathway of leukotriene metabolism.

19. The method of claim 18, wherein the leukotriene modifier inhibits the activity of 5-lipoxygenase.

20. The method of claim 18, wherein the leukotriene modifier inhibits the activity of 5-lipoxygenase-activating protein (FLAP).

21. The method of claim 18, wherein the leukotriene modifier is Zileuton.

22. The method of claim 14, wherein the donor has, or is at risk of developing a chronic obstructive disease of the airways.

23. The method of claim 14, wherein the step of determining the level of leukotriene in the sample comprises the method of claim 1.

24. The method of claim 22, wherein the chronic obstructive disease of the airways is asthma and the level of LTE₄ in the sample is at or greater than about 80 pg/mg of creatinine.

25. A method for diagnosing a chronic obstructive disease of the airways in a donor,
comprising obtaining a sample from the donor and determining the level of leukotriene in the sample selected from the group consisting of LTA₄, LTB₄, LTC₄, LTD₄, LTE₄, and LTF₄, and combinations thereof,
wherein presence of leukotriene in the sample at an elevated level as compared to a baseline level established from a control sample, identifies the donor as having or likely to develop the chronic obstructive disease of the airways.

26. The method of claim 25, wherein the step of determining the level of leukotriene in the sample comprises determining the level of LTE₄.

27. The method of claim 26, wherein the step of determining the level of LTE₄ in the sample comprises the method of claim 1.

28. The method of claim 26, wherein the chronic obstructive disease of the airways is asthma and the level of LTE₄ in the sample is at or greater than about 30 pg/mg of creatinine.

29. A method for predicting the risk for a donor of exacerbation due to a chronic obstructive disease of the airways, comprising
obtaining a sample from the donor and determining the level of leukotriene in the sample selected from the group consisting of LTA₄, LTB₄, LTC₄, LTD₄, LTE₄, and LTF₄, and combinations thereof,
wherein presence of leukotriene in the sample at an elevated level as compared to a baseline level established from a control sample, identifies the donor as at risk of exacerbation due to the chronic obstructive disease.

30. The method of claim 29, wherein the step of determining the level of leukotriene in the sample comprises determining the level of LTE₄.

31. The method of claim 30, wherein the step of determining the level of LTE₄ in the sample comprises the method of claim 1.

32. The method of claim 30, wherein the chronic obstructive disease of the airways is asthma and the level of LTE₄ in the sample is at or greater than about 90 pg/mg of creatinine.
